(19) United States
(12) Patent Application Publication
(10) Pub. No.: US 2022/0079948 A1
(43) Pub. Date: Mar. 17, 2022

(54) COMPOSITIONS AND METHODS FOR SUPPRESSING AND/OR TREATING HIV-INFECTION AND/OR A RELATED CLINICAL CONDITION THEREOF

(71) Applicant: Indiana University Research And Technology Corporation, Indianapolis, IN (US)

(72) Inventors: Matthias Alexander Clauss, Indianapolis, IN (US); Bernhard F. Maier, Carmel, IN (US); Sarvesh Chelvanambi, Boston, MA (US)

(73) Assignee: Indiana University Research And Technology Corporation, Indianapolis, IN (US)

(21) Appl. No.: 17/416,947

(22) PCT Filed: Dec. 20, 2019
(86) PCT No.: PCT/US2019/067936

§ 371 Date: Jun. 21, 2021

Related U.S. Application Data
(60) Provisional application No. 62/784,047, filed on Dec. 21, 2018.

(51) Int. Cl. A61K 31/519; A61K 45/06; A61K 31/138; G01N 33/577

(52) U.S. Cl. CPC A61K 31/519; A61K 45/06; G01N 2800/24; G01N 33/577; A61K 31/138

(57) ABSTRACT

Therapeutic compositions comprising one or more agents that inhibit the Nef-PAK2 interface, and methods of administering those therapeutic compositions to model, treat, reduce resistance to treatment, prevent, and diagnose a condition/disease associated with HIV-infection or a related clinical condition thereof, are disclosed.

### STATEMENT OF GOVERNMENTAL RIGHTS

This invention was made with government support under HL129843 awarded by National Institutes of Health. The government has certain rights in the invention.

### FIELD

Various aspects and embodiments disclosed herein relate generally to the modelling, treatment, reducing resistance to the treatment, prevention, and diagnosis of a condition/disease associated with human immunodeficiency virus (HIV)-infection or a related clinical condition thereof. Embodiments include compositions and methods for treating the condition/disease, comprising providing to a subject at least one therapeutically effective dose of a composition disclosed herein. Other embodiments include methods for altering and/or suppressing the activity of at least one Nef-PAK2 interface in a subject.

### BACKGROUND

After an HIV infection, the lymphatic reservoir includes one or more subsets of HIV-infected, but latent CD4⁺ T lymphocytes, dendritic cells, and cells of monocyte-macrophage lineage. These cells harbor transcriptionally silent but replication-competent HIV DNA, beyond the reach of hot immune responses and antiretroviral therapy (ART). Accordingly, a major roadblock for HIV eradication is in part due to the ability of latent, HIV-infected T cells that hide out in lymphatic reservoirs where delivery of anti-retroviral drugs is severely limited. “Shock and kill” therapy of latent reservoirs of HIV-infected cells has been suggested as one major step towards curing diseases/conditions associated with an HIV infection. However, it is not clear whether all latent HIV-infected T cells will be treated and/or killed providing that intrinsic pro-lytic and apoptotic viral activities are limited to activated T cells.

In addition, HIV specific cytotoxic T cells, which could contribute to killing, are not present in follicular lymph node structures harboring latently infected memory T cells, as these comprise immune-privileged areas. Further, chronic HIV infection can cause retention of lymphocytes. It is presently believed that this retention is likely caused by the altered lymphocyte trafficking due in part to virally-induced changes in chemokine receptors and adhesion molecules of T lymphocytes. Due to the complexity of an HIV infection, development of a new treatment regimen that would enhance the effect of HIV treatment on a patient is much needed. More specifically, a need exists for the development of a therapeutic to effectively treat the HIV-infected, but latent CD4⁺ T lymphocytes, dendritic cells, and/or cells of monocyte-macrophage lineage that exist in the lymphatic reservoir of HIV-infected subjects.

### SUMMARY

Embodiments of the instant application relate to compositions and methods for treating a condition and/or disease associated with HIV-infection or a related clinical condition in a subject. In certain embodiments, the compositions and methods disclosed herein concern suppression of one or more side effects of a therapeutic regime. Other embodiments relate to compositions and methods for treating a subject diagnosed with a disease or having a condition contributed to human immunodeficiency virus (HIV) infection, acquired immunodeficiency syndrome (AIDS), and/or any other conditions associated with, induced by, or that are already resistant to antiretroviral therapy (ART) or highly active antiretroviral therapy (HAART).

In a first aspect, compositions disclosed herein comprise at least one agent that inhibits the Nef-PAK2 interface (the interface between Negative Regulatory Factor (Nef) and p21 activated protein kinase 2) and/or Nef-AP2 interface (Nef and clarthrin adaptor complex AP-2) in a subject. In some embodiments, the at least one agent that inhibits Nef-PAK2 interface comprises at least one Nef inhibitor, at least one PAK2 inhibitor, or a combination thereof. In certain embodiments, the composition further comprises at least one antiretroviral agent. Antiretroviral agents can include, but are not limited to, zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir lopinavir, indinavir, nelfinavir, amprenavir, ritonavir, darunavir, atazanavir, bevirimat, vivecon, or any combination thereof.

In accordance with these embodiments, the composition can further comprise at least one agent that inhibits β-arrestin recruitment to at least one sphingosine-1-phosphate receptor. Sphingosine-1-phosphate receptors can include, but are not limited to, Sphingosine-1-Phosphate Receptor 1 (S1P1), Sphingosine-1-Phosphate Receptor 2 (S1P2), Sphingosine-1-Phosphate Receptor 3 (S1P3), Sphingosine-1-Phosphate Receptor 4 (S1P4), and/or Sphingosine- 1-Phosphate Receptor 5 (S1P5). In certain embodiments, the compositions disclosed herein comprise at least one PAK2 inhibitor selected from FRAX1036, FRAX597, miR-23b, and/or miR-137. In some embodiments, the compositions disclosed herein comprise at least one phosphatase involves in lysosomal degradation. The at least one phosphatase includes, but is not limited to, protein phosphatase 2 alpha (PP2α).

In a second aspect, methods disclosed herein include a method of treating a clinical condition, comprising administering to a subject at least one therapeutically effective dose of any of the compositions disclosed herein. The subject can be diagnosed with a clinical condition selected from and/or comprising HIV infection, acquired immunodeficiency syndrome (AIDS), and/or any other conditions associated with, induced by, or that are already resistant to antiretroviral therapy (ART) or highly active antiretroviral therapy (HAART). In certain embodiments, the methods disclosed herein further comprise administering to the subject at plurality of therapeutically effective doses of any of the compositions disclosed herein.

In a third aspect, methods provided by the present application reduce and/or suppress a side effect of a therapeutic regime, the methods comprising administering to a subject at least one therapeutically effective dose of at least one agent that inhibits Nef-PAK2 interface in a subject; wherein the subject has received at least one therapeutic regime selected from surgery, antiretroviral therapy (ART), highly active antiretroviral therapy (HAART) and combinations thereof, and wherein the subject experiences at least one side effect as a consequence of the therapeutic regime. Consistent with these embodiments, side effects can include, but are not limited to, drug-resistance, relapse, retention of HIV-infected lymphocytes, generation of a viral reservoir, or any combination thereof.

### BRIEF DESCRIPTION OF THE DRAWINGS

The following drawings form part of the present specification and are included to further demonstrate certain embodiments. Some embodiments may be better understood by reference to one or more of these drawings alone or in combination with the detailed description of specific embodiments presented.

FIG. 1A. Graph illustrating the effect of the PAK2 inhibitor FRAX597 (5 μM) on the level of S1P1 surface expression in either SupT1 wildtype (SupT1) or cells stably expressing tamoxifen-inducible HIV-1SF2-Nef-estrogen receptor construct (Nef-ER).

FIG. 1B. Graph illustrating the effect of the PAK2 inhibitor FRAX597 (5 μM) on migration of SupT1 wildtype (SupT1) or cells stably expressing tamoxifen-inducible HIV-1SF2-Nef-estrogen receptor construct (Nef-ER) through collagen I coated transwell chamber.

FIG. 1C. Schematic diagram illustrating targeted retention of HIV-Nef containing active HIV reservoir T cells in sanctuary lymphatic organs.

FIG. 1D. Graph illustrating the effect of the PAK2 inhibitor FRAX597 (5 μM) on the level of S1P1 surface expression in either SupT1 wildtype (SupT1) or cells stably expressing tamoxifen-inducible HIV-1SF2-Nef-estrogen receptor construct (Nef-ER).

FIG. 2A. Graph illustrating dose response of sphingosine-1-phosphate (S1P)-mediated β-arrestin recruitment, β-arrestin recruitment was measured using the TANGO® assay 24 hours after transfection with TANGO-S1P1 Shown are the effects of increasing concentration of S1P when added to cells co-transfected with mock plasmid (black circle, closed black line), mock plasmid with 1 μM fingolimod (FTY720) as a positive control for increased β-arrestin recruitment (black square, dotted line) and Nef plasmid (red triangle, red line).

FIG. 2B. Graph illustrating the effect of Nef or the PAK2 activation deficient Nef-mutant (F195R) on sphingosine-1-phosphate (S1P)-mediated β-arrestin recruitment. β-arrestin recruitment was measured using the TANGO® assay 48 hours after concomitant transfection of TANGO-S1P1 with mock, Nef or the PAK2 activation deficient Nef-mutant (F195R).

FIG. 2C. Western blot illustrating the effect of Nef or Nef-mutant (F195R) on stability of S1P1 after the treatment with S1P (10 μM) and cycloheximide (100 μM) for indicated time points. Stability of S1P1 was assessed in Western blot analysis of HTLA cells after various times of cycloheximide treatment (50 μM).

FIG. 2D. Schematic diagram illustrating the mechanism of HIV-Nef-PAK2 mediated increase in β-arrestin recruitment and clathrin coated pit formation that leads to augmented S1P1-endocytosis and subsequent degradation in lysosomes (solid arrows) as well as decreased recycling (dotted arrows). CCP, clathrin coated pit.

FIG. 3. Graph illustrating the effect of HIV-Nef on S1P1 surface expression in peripheral blood monocytes from HIV negative donors when treated with Nef containing extracellular vesicles for approximately 16 hours. S1P1 receptor was determined by flow using specific antibodies. The means were compared using Student's t test, a p<0.05 was considered significant, n=4.

### DEFINITIONS

“About” refers to a range of values plus or minus 10 percent, e.g. about 1.0 encompasses values from 0.9 to 1.1.

“Antiretroviral therapy agents” or “antiretroviral agents” can include, but are not limited to, zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir lopinavir, indinavir, nelfinavir, amprenavir, ritonavir, darunavir, atazanavir, bevirimat, and vivecon, or any combination thereof.

“Inhibitor of Nef-PAK2 interface” refers to an agent that alters the function and/or activity of the Nef-PAK2 interface or induces conformational changes in the Nef-PAK2 interface. Examples of inhibitors of Nef-PAK2 interface include, but are not limited to, agents that alter association/dissociation between Nef and PAK2 and/or agents that inhibit Nef-PAK2 complex assembly/function.

“Nef-PAK2 interface” refers to an interaction and/or association between HIV-Nef (or “Nef”—Negative Regulatory Factor) and PAK2 (p21 activated kinase 2), which can induce biological activities known in the art. The interactions and/or associations can be physical or chemical interactions that would activate a Nef-PAK2 pathway within a subject. Nef-PAK2 interface can be present in a form of a complex.

“Nef-AP2 interface” refers to an interaction and/or association between HIV-Nef and AP2, which can induce biological activities known in the art. The interactions and/or associations can be physical or chemical interactions that would activate a Nef-AP2 pathway within a subject. Nef-AP2 interface can be present in a form of a complex.

“PAK2-AP2-β-arrestin interface” refers to an interaction and/or association between PAK2 (p21 activated kinase 2), AP2, and β-arrestin, which can induce biological activities known in the art. The interactions and/or associations can be physical or chemical interactions that would activate a PAK2/AP2/β-arrestin pathway within a subject. PAK2-AP2-β-arrestin interface can be present in a form of a complex.

“Pharmaceutically acceptable” refers to approved or approvable by a regulatory agency of a government, such as the U.S. FDA or the EMA, or listed in the U.S. Pharmacopoeia or other generally recognized pharmacopoeia for use in mammals and/or animals, and more particularly in humans.

“Pharmaceutically acceptable vehicle” or “pharmaceutically acceptable carrier,” unless stated or implied otherwise, is used herein to describe any ingredient other than the active component(s) that can be included in a formulation. The choice of carrier will to a large extent depend on factors such as the mode of administration, the effect of the carrier on solubility and stability, and the nature of the dosage form.

“Pharmaceutical composition” refers to a therapeutically active inhibitor of Nef-PAK2 interface, inhibitor of PAK2-AP2-β-arrestin interface, or a therapeutically active inhibitor of β-arrestin recruitment, and at least one pharmaceutically acceptable vehicle/carrier, with which the inhibitor of Nef-PAK2 interface, inhibitor of PAK2-AP2-β-arrestin interface, or inhibitor of β-arrestin recruitment is administered to a subject.

“Sphingosine-1-Phosphate (S1P) Receptor” can include, but is not limited to, Sphingosine-1-Phosphate Receptor 1 (S1P1), Sphingosine-1-Phosphate Receptor 2 (S1P2), Sphingosine-1-Phosphate Receptor 3 (S1P3), Sphingosine-1-Phosphate Receptor 4 (S1P4), and Sphingosine-1-Phosphate Receptor 5 (S1P5),

“Sphingosine-1-phosphate receptor modulators” can include, but are not limited to, fingolimod, ozanimod, ponesimod, and laquinimod.

“Subject” refers to a mammal, for example, a human.

“Therapeutically effective amount” refers to the amount of an inhibitor of Nef-PAK2 interface or inhibitor of β-arrestin recruitment that, when administered to a subject for treating a disease, or at least one of the clinical symptoms of a disease, is sufficient to affect such treatment of the disease or symptom thereof. The “therapeutically effective amount” can vary depending, for example, on the inhibitor of Nef-PAK2 interface, inhibitor of PAK2-AP2-β-arrestin interface, or inhibitor of β-arrestin recruitment, the disease and/or symptoms of the disease, severity of the disease and/or symptoms of the disease or disorder, the age, weight, and/or health of the subject to be treated, and the judgment of the prescribing physician.

“Therapeutically effective dose” refers to a dose that provides effective treatment of a disease or disorder in a subject. A therapeutically effective dose can vary from compound to compound, and from subject to subject, and can depend upon factors such as the condition of the subject and the route of delivery.

“Therapeutic regime(s)” and/or “therapeutic regimen(s)” include, but are not limited to, surgery, antiretroviral therapy (ART), and/or highly active antiretroviral therapy (HAART).

“Treat,” “treating” or “treatment” of any disease refers to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. “Treat,” “treating” or “treatment” also refers to inhibiting the disease, either physically, (e.g., stabilization of a discernible symptom), physiologically, (e.g., stabilization of a physical parameter), or both, and to inhibiting at least one physical parameter that can or cannot be discernible to the subject. In certain embodiments, “treat,” “treating” or “treatment” refers to delaying the onset of the disease or at least one or more symptoms thereof in a subject which can be exposed to or predisposed to a disease even though that subject does not yet experience or display symptoms of the disease.

### DETAILED DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

A major roadblock for HIV eradication is in part due to the ability of latent, HIV-infected T cells that hide out in lymphatic reservoirs where delivery of anti-retroviral drugs is severely limited. “Shock and kill” therapy of latent reservoirs of HIV-infected cells has been suggested as one major step towards curing diseases/conditions associated with an HIV infection. However, it is not clear whether all latent HIV-infected T cells will be treated and/or killed providing that intrinsic pro-lytic and apoptotic viral activities are limited to activated T cells.

The mechanisms for HIV-mediated maintenance of viral reservoirs in lymph nodes even under antiretroviral therapy are not well understood. S1P receptor 1 (S1P1) signaling is a key mechanism for lymph node egress and trafficking of lymphocytes. The instant disclosure provides a novel function of Nef that leads to downregulation and degradation of S1P1. To address the mechanism of S1P1 downregulation and to identify potential targets for intervention, S1P1 stability, β-arrestin recruitment and steady state surface expression of S1P1 upon Nef expression were assessed. Results disclosed herein emphasize a specific Nef-PAK2 interface as a therapeutic target for reconstitution of S1P1 signaling.

The present disclosure provides, inter cilia, a discovery platform for developing therapeutic inhibitors of a specific HIV-Nef signaling pathway that affects S1P receptor 1 (S1P1), a G-Protein coupled receptor (GPCR) that is essential for T lymphocytes, macrophages and dendritic cells to egress from lymphoid tissues and possibly other confined spaces in various tissues, for example liver and others. HIV-Nef increases beta arrestin recruitment to S1P1 and lysosomal degradation of S1P1 by preventing its recycling to the cell surface. This mechanism of S1P1 downregulation and degradation involves the interaction of Nef with Pak2 that leads to the autocatalytic phosphorylation and activation of Pak2 that is crucial for the induction of endocytosis by engaging cytoskeletal components. By identifying this Nef induced mechanism of S1P1 downregulation, the present disclosure provides a platform for screening compound libraries for inhibitors of the specific interaction of HIV-1 Nef with Pak2 by binding to the Nef-Pak2 interface that involves the phenylalanine residue at position 195 of HIV-1 Nef.

Briefly, the discovery platform disclosed herein can comprise a stepwise process using transfected and/or transduced T cells. For example, compounds or libraries of compounds selected in silico can be examined to determine S1P1 downreguiation using TANGO® assay or Western blotting analysis in the presence or absence of cycloheximide, Alternatively, S1P1 signaling can be assessed by measuring cAMP levels and/or Erk phosphorylation using Western blotting or immunostaining. In parallel, a selected panel of compounds can be examined for their ability to stabilize and maintain surface expression, e.g., by flow cytometry. Further, T cell migration assay can be utilized to measure the number of T cells responding to gradient of S1P through collagen coated transwell filters or endothelial monolayers. Selected compounds can be tested in in vivo systems where HIV-Nef is conditionally expressed in CD4 positive cell; for example, Nef expression can be measured by bicistronic eGFP expression. Nef can be expressed conditionally for 1, 2, 3, 4, and/or 5 days and egressed into the blood stream. This effect can be measured using, for example, flow cytometry.

Embodiments disclosed herein relate to compositions and methods for treating a condition and/or disease associated with an HIV-infection or a related clinical condition in a subject. In certain embodiments, compositions and methods disclosed herein concern suppression of a side effect of a therapeutic regime. Other embodiments relate to compositions and methods for treating a subject diagnosed with a disease or having a condition contributed to human immunodeficiency virus (HIV) infection, acquired immunodeficiency syndrome (AIDS), and/or any other conditions associated with, induced by, or that are already resistant to antiretroviral therapy (ART) or highly active antiretroviral therapy (HAART).

In some embodiments, compositions disclosed herein comprise at least one agent that inhibits Nef-PAK2 interface in a subject. Consistent with these embodiments, the at least one agent that inhibits Nef-PAK2 interface comprises preferably direct binding competition (such as inhibitors are not known), at least one Nef inhibitor which may affect Nef-PAK2 activation indirectly such as the mainly SH3 interfering “neffin” (see, e.g., Jarviluoma A, et al., PLoS One. 2012; 7 (7):e40331. doi: 10.1371/journal.pone.0040331. PubMed PMID: 22792285; PubMed Central PMCID: PMC3390362 and Bouchet J, et al., J. Virol. 2012, 86 (9):4856-67), at least one PAK2 inhibitor, or a combination thereof. PAK2 inhibitors can include, but are not limited to, FRAX1036, FRAX597, miR-23b, and/or miR-137, or any combination thereof. In some embodiments, the at least one agent that inhibits Nef-PAK2 interface can disrupt conformation of the Nef-PAK2 interface physically and/or chemically.

In some embodiments, the composition can further comprise at least one antiretroviral agent. Antiretroviral agents can include, but are not limited to, zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir lopinavir, indinavir, nelfinavir, amprenavir, ritonavir, darunavir, atazanavir, bevirimat, and/or vivecon, or any combination thereof.

In certain embodiments, the composition comprises at least one agent that inhibits Nef-PAK2 interface in a subject and at least one antiretroviral agent. The at least one agent that inhibits Nef-PAK2 interface can comprise at least one Nef inhibitor, at least one PAK2 inhibitor, or a combination thereof, as noted above, and the antiretroviral agent can comprise the agent(s) noted above.

In some embodiments, the composition comprises at least one agent that inhibits Nef-AP2 interface in a subject and at least one antiretroviral agent. The at least one agent that inhibits Nef-AP2 interface can comprise at least one Nef inhibitor, at least one AP2 inhibitor, or a combination thereof, as noted above, and the antiretroviral agent can comprise the agents) noted above.

In some embodiments, the composition further includes at least one agent that inhibits β-arrestin recruitment to at least one sphingosine-1-phosphate receptor. Sphingosine-1-phosphate receptors can include, but are not limited to, Sphingosine-1-Phosphate Receptor 1 (S1P1), Sphingosine-1-Phosphate Receptor 2 (S1P2), Sphingosine-1-Phosphate Receptor 3 (S1P3), Sphingosine-1-Phosphate Receptor 4 (S1P4), and/or Sphingosine-1-Phosphate Receptor 5 (S1P5).

In certain embodiments, the composition disclosed herein can further include at least one PAK2 inhibitor selected from FRAX1036, FRAX597, miR-23b, and/or miR-137. In some embodiments, the compositions disclosed herein comprise at least one phosphatase involves in lysosomal degradation. The at least one phosphatase includes, but is not limited to, PP2α.

In some embodiments, the composition further includes at least one agent that inhibits or interferes a PAK2-AP2-β-arrestin interface. The at least one agent that inhibits or interferes a PAK2-AP2-β-arrestin interface can include, but is not limited to, barbardin (see, e.g., Beautrait A, et al., Nature Comm. 2017; 8:15054).

In any one of the embodiments disclosed herein, the subject comprises a human, an animal, a livestock, a tissue, or a cell. In some embodiments, the subject has been infected by human immunodeficiency virus (HIV) and has been treated with the at least one antiretroviral agent.

### Pharmaceutical Compositions

Pharmaceutical compositions provided by the present disclosure can comprise a therapeutically effective amount of one or more compositions disclosed herein, together with a suitable amount of one or more pharmaceutically acceptable vehicles to provide a composition for proper administration to a subject. Suitable pharmaceutical vehicles are described in the art.

Pharmaceutical compositions of the present disclosure suitable for oral administration can be presented as discrete units, such as a capsule, cachet, tablet, or lozenge, each containing a predetermined amount of the active ingredient; as a powder or granules; as a solution or a suspension in an aqueous liquid or non-aqueous liquid such as a syrup, elixir or a draught, or as an oil-in-water liquid emulsion or a water-in-oil liquid emulsion. The composition can also be presented as a bolus, electuary or paste. A tablet can be made by compressing or moulding the active ingredient with the pharmaceutically acceptable carrier. Compressed tablets can be prepared by compressing in a suitable machine the active ingredient in a free-flowing form, such as a powder or granules, in admixture with, for example, a binding agent, an inert diluent, a lubricating agent, a disintegrating and/or a surface-active agent. Moulded tablets can be prepared by moulding in a suitable machine a mixture of the powdered active ingredient moistened with an inert liquid diluent. The tablets can optionally be coated or scored and can be formulated to provide slow or controlled release of the active ingredient.

Pharmaceutical compositions of the present disclosure suitable for parenteral administration include aqueous and non-aqueous sterile injection solutions, and can also include an antioxidant, buffer, a bacteriostat and a solution which renders the composition isotonic with the blood of the recipient, and aqueous and non-aqueous sterile suspensions which can contain, for example, a suspending agent and a thickening agent. The formulations can be presented in a single unit-dose or multi-dose containers and can be stored in a lyophilized condition requiring the addition of a sterile liquid carrier prior to use.

In some embodiments, the composition can contain pharmaceutically acceptable lubricant(s). The pharmaceutically acceptable lubricant(s) prevent the components of the pharmaceutical composition from clumping together and from sticking to the pellet press that generates the disclosed compositions. The lubricant(s) also ensure that the formation of the pellet, as well as its ejection from the pellet press, occurs with low friction between the composition and the wall of the die press. In some embodiments, the lubricant(s) are added to a pharmaceutical composition to improve processing characteristics, for example to help increase the flexibility of the compositions, thereby reducing breakage.

The type of lubricant that can be used in the disclosed pharmaceutical compositions can vary. In some embodiments, the pharmaceutically acceptable lubricant is selected from talc, silica, vegetable stearin, magnesium stearate, stearic acid, calcium stearate, glyceryl behenate, glyceryl monostearate, glyceryl palmitostearate, mineral oil, polyethylene glycol, sodium stearyl fumarate, sodium lauryl sulfate, vegetable oil, zinc stearate, and combinations thereof. In some embodiments, the pharmaceutically acceptable lubricant is stearic acid.

The type of vehicles that can be used in the disclosed pharmaceutical compositions can vary. In some embodiments, the pharmaceutically acceptable vehicles are selected from binders, fillers and combinations thereof. In some embodiments, the pharmaceutically acceptable vehicle is selected from ascorbic acid, polyvinylpyrrolidone, polyvinylpyrrolidone K-30 (povidone K-30), glyceryl monostearate (GMS) or glyceryl monostearate salts, glyceryl behenate, glyceryl palmitostearate, hydroxypropyl cellulose, hydroxypropyl methylcellulose, methylcellulose, hydroxyethyl cellulose, sugars, dextran, cornstarch, dibasic calcium phosphate, dibasic calcium phosphate dihydrate, calcium sulfate, dicalcium phosphate, tricalcium phosphate, lactose, cellulose including microcrystalline cellulose, mannitol, sodium chloride, dry starch, pregelatinized starch, compressible sugar, mannitol, lactose monohydrate, starch, dibasic calcium phosphate dihydrate, calcium sulfate, dicalcium phosphate, tricalcium phosphate, powdered cellulose, microcrystalline cellulose, lactose, glucose, fructose, sucrose, mannose, dextrose, galactose, the corresponding sugar alcohols and other sugar alcohols, such as mannitol, sorbitol, xylitol, and combinations of any of the foregoing. In some embodiments, the pharmaceutically acceptable vehicle is polyvinylpyrrolidone K-30, also known as povidone K-30. In some embodiments, the pharmaceutically acceptable vehicle is polyvinylpyrrolidone K-30, also known as povidone K-30, having an average molecular weight of MW of 40,000 (CAS 9003-39-8). In some embodiments, the pharmaceutically acceptable vehicle is selected from glyceryl monostearate (GMS) or glyceryl monostearate salts, glyceryl behenate and glyceryl palmitostearate. In some embodiments, the pharmaceutically acceptable vehicle is glyceryl monostearate (GMS) or glyceryl monostearate salts. In some embodiments, the pharmaceutically acceptable vehicle is glyceryl behenate. In some embodiments, the pharmaceutically acceptable vehicle is glyceryl palmitostearate.

In some embodiments, the antioxidants prevent oxidation of the other components of the disclosed compositions. Oxidation can occur, for example, during sterilization where free radicals are generated. Addition of the antioxidants, or free radical scavengers, significantly reduces oxidation and makes the composition more pharmaceutically acceptable for use in subjects.

The type of antioxidants that can be used in the disclosed pharmaceutical compositions can vary. In some embodiments, the antioxidant is selected from methyl paraben and salts thereof, propyl paraben and salts thereof, vitamin E, vitamin E TPGS, propyl gallate, sulfites, ascorbic acid (aka L-ascorbic acid, also including the L-enantiomer of ascorbic acid, vitamin C), sodium benzoate, citric acid, cyclodextrins, peroxide scavengers, benzoic acid, ethylenediaminetetraacetic acid (EDTA) and salts thereof, chain terminators (e.g., thiols and phenols), butylated hydroxytoluene (BHT), butylated hydroxyanisole (BHA), and combinations thereof.

### Uses

The methods and compositions disclosed herein can be used to treat subjects suffering from diseases, disorders, conditions, and symptoms for which inhibitors of Nef-PAK2 interface, Nef-AP2 interface, PAK2-AP2-β-arrestin interface, and/or β-arrestin recruitment are known to provide or are later found to provide therapeutic benefit.

In some embodiments, methods disclosed herein include a method of treating a clinical condition, comprising administering to a subject at least one therapeutically effective dose of any of the compositions disclosed herein. The subject can be diagnosed with a clinical condition selected from and/or comprising immunodeficiency virus (HIV) infection, acquired immunodeficiency syndrome (AIDS), and/or any other conditions associated with, induced by, or that are already resistant to anti retroviral therapy (ART) or highly active anti retroviral therapy (HAART). In certain embodiments, the methods disclosed herein further comprise administering to the subject at least one additional therapeutically effective dose of any of the compositions disclosed herein. In some embodiments, the at least one therapeutically effective dose of any of the compositions disclosed herein can be administered orally, parenterally, intravenously, by inhalation and/or transdermally.

Yet other embodiments can include methods for reducing a side effect of a therapeutic regime, comprising administering to a subject at least one therapeutically effective dose of at least one agent that inhibits Nef-PAK2 interface and/or Nef-AP2 interface in a subject; wherein the subject has received at least one therapeutic regime comprising surgery, antiretroviral therapy (ART), and/or highly active antiretroviral therapy (HAART), and wherein the subject experiences at least one side effect derived from the therapeutic regime. Consistent with these embodiments, side effects can include, but are not limited to, drug-resistance, relapse, retention of HIV-infected lymphocytes, or generation of a viral reservoir.

In accordance with these embodiments, the at least one therapeutically effective dose of at least one agent that inhibits Nef-PAK2 interface and/or Nef-AP2 interface comprises at least one Nef inhibitor, at least one PAK2 inhibitor, at least one AP2 inhibitor, and/or at least one agent that inhibits recruitment of β-arrestin to at least one sphingosine-1-phosphate receptor, or a combination thereof. In certain embodiments, the at least one agent that inhibits Nef-PAK2 interface and/or Nef-AP2 interface comprises at least one phosphatase that involves in lysosomal degradation and/or at least one agent that inhibits recruitment of β-arrestin to at least one sphingosine-1-phosphate receptor. The at least one phosphatase that involves in lysosomal degradation includes, but is not limited to, PP2α. Sphingosine-1-phosphate receptors can include, but are not limited to, Sphingosine-1-Phosphate Receptor 1 (S1P1), Sphingosine-1-Phosphate Receptor 2 (S1P2), Sphingosine-1-Phosphate Receptor 3 (S1P3), Sphingosine-1-Phosphate Receptor 4 (S1P4), and/or Sphingosine-1-Phosphate Receptor 5 (S1P5).

In some embodiments, the subject has been infected by human immunodeficiency virus (HIV) and the subject has been treated with the at least one antiretroviral agent.

### Kits

In a further aspect, kits are provided by the present disclosure, such kits comprising: one or more pharmaceutical compositions, each composition sterilized within a container, means for administration of the pharmaceutical compositions to a subject, and instructions for use.

Some embodiments include kits for carrying out the methods disclosed herein. Such kits typically comprise two or more components required for treating a clinical condition. Components of the kit include, but are not limited to, one or more of agents/compositions disclosed herein, reagents, containers, equipment and/or instructions for using the kit. Accordingly, the compositions and methods described herein can be performed by utilizing pre-packaged kits disclosed herein.

### EXAMPLES

The following examples illustrate various aspects of the disclosure. It will be apparent to those skilled in the art that many modifications, both to materials and methods, can be practiced without departing from the scope of the disclosure.

S1P1 cell surface staining. For assessing Nef-dependent S1P1 regulations, stably transfected T cells (SupT) resulting in estrogen inducible Nef expression (Nef-ER) were used (see Lee J. H. et al. HIV Activates the Tyrosine Kinase Hck to Secrete ADAM Protease-Containing Extracellular Vesicles. EBIOMEDICINE 2018, 28:151-161). Upon treatment with tamoxifen (1 μM; established using CD4 downregulation) for 16 hours, fluorescence cytometry analysis was performed with an anti-S1P1 monoclonal antibody coupled to eFluor 660 (SW4GYPP, EBIOSCIENCE™).

Migration assay. 4-Hydroxy tamoxifen (4HT) activated SupT1 control and Nef-ER cells were labeled with 2.5 uM Calcein-AM, washed using centrifugation and resuspended in phenol red free RPMI containing 0.1% fatty acid free BSA. These cells were loaded on the top chamber of collagen I-coated 4 micron transwell chambers as previously described (see, e.g., Iino J. et al. Platelet-derived sphingosine 1-phosphate induces migration of Jurkat T cells. LIPIDS HEALTH DIS 2014, 13:150). Briefly, cells were allowed to transmigrate,

Tango assay and Transfection with Nef and S1P1: The S1P1-Tango plasmid coding for S1P1 fused on the C-terminus to the Tet-activator (tTA) via a linked TEV-protease recognition site (Addgene) was co-transfected into HTLA cells harboring a tet-luciferase gene a gene for a arrestin/Tev protease fusion gene that were seeded in 96-well plates (4×10⁴ cells/well) by calcium phosphate co-precipitation and incubated overnight. After media change to DMEM with 1% FBS cells were incubated for further 6 hours after which cells were exposed to titrated concentrations of S1P. After 16 hrs, media was replaced with 50 μL of Bright-Glo luciferase substrate. Plates were incubated for 5 minutes at 37° C. before luminescence reading in a plate reader.

Tango assay and Transfection with Nef and S1P1. The S1P1-Tango plasmid coding for S1P1 fused on the C-terminus to the Tet-activator (tTA) via a linked TEV-protease recognition site (Addgene) was co-transfected into HTLA cells seeded in 96-well plates (4×10⁴ cells/well) by calcium phosphate co-precipitation and incubated overnight. After media change to DMEM with 1% FBS cells were incubated for further 6 hours after which cells were exposed to titrated concentrations of S1P. After 16 hrs, media was replaced with 50 μL of Bright-Glo luciferase substrate. Plates were incubated for 5 minutes at 37° C. before luminescence reading in a plate reader.

Western blotting. Western blotting: After 2 washes with ice-cold PBS, cells were lysed in 4° C. RIPA buffer (Thermo-Fisher) containing protease and phosphatase inhibitor cocktails (Roche) and Benzonase (0.5 μl/ml lysis buffer). Protein concentration was determined with a modified Lowry assay (Biorad) and 20 ug protein per sample was separated by Tris/glycine SDS-PAGE on 4-20% gradient gels. After electrophoretic protein transfer, PVDF Immobilon-F membranes were blocked with Licor blocking buffer followed by detection of S1P1 with a fluorophore-conjugated antibody (SW4GYPP, eBioscience). Blots were scanned on a Licor Odyssey scanner.

To explain how HIV in patients treated successfully with combined antiretroviral therapy (ART) can confine latently infected cells in lymph nodes, HIV-Nef (“Nef”) induced S1P1 downregulation was examined. Nef is an HIV encoded protein which persists in extracellular vesicles (EV) in circulating blood and in peripheral blood mononuclear cells of HIV-infected individuals.

S1P1 has been studied for the development of autoimmune-suppressive drugs such as the compound FTY720 better known as fingolimod, an FDA approved drug to treat multiple sclerosis relapses. FTY720 has been reported to cause lymphocyte retention in lymphoid structures through S1P1 downregulation.

The present disclosure provides data showing a previously unknown function of HIV-Nef that downregulates from the cell surface and leads to degradation of S1P receptor 1 (S1P1) in human T cells. To address the mechanism of this downregulation, the present inventors employed the Tango-HTLA cell assay, which allowed them to measure β-arrestin recruitment by ligand activated S1P1. In addition, surface expression and stability of S1P1. was characterized by cytofluorometry and Western blot analysis after cycloheximide treatment. Overexpression of wildtype HIV-Nef in HTLA cells caused β-arrestin mediated S1P1 endoytosis and degradation, which was not observed with overexpression of the PAK2 activation mutant HIV-Nef (F195R). These results suggest that HIV can inactivate a signaling pathway essential for T cell egress from lymphoid tissues, and thus contribute to increased HIV maintenance in reservoirs.

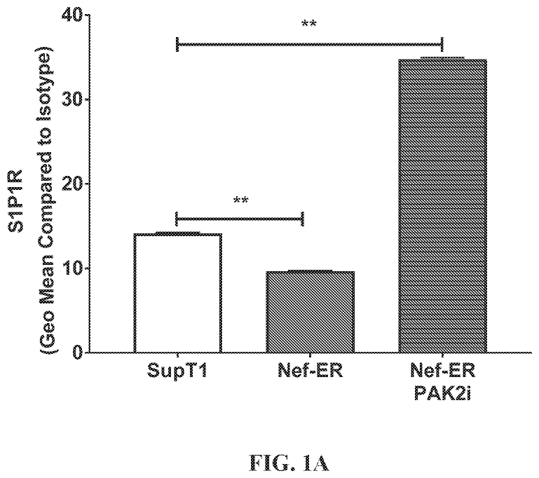

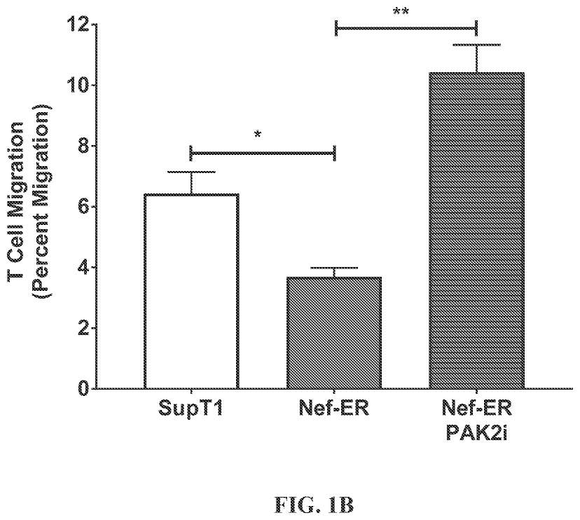

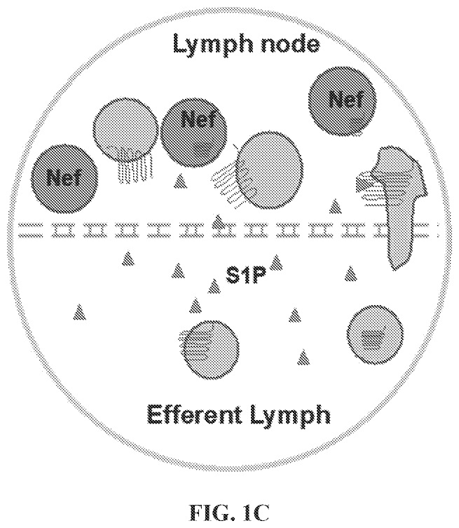

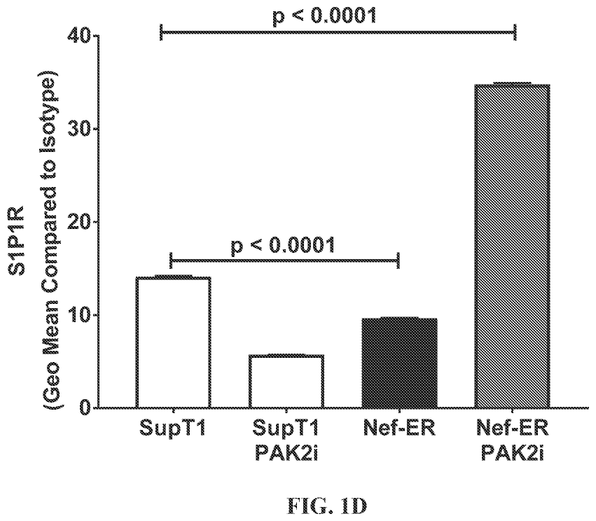

HIV-Nef Leads to S1P1 Downregulation and Impaired Migration of T cells to S1P1 Gradients. The effect of Nef on regulation of S1P1 surface expression was examined using a recombinant human T cell line (SupT1), which stably expresses a tamoxifen inducible Nef (Nef-ER). Upon treatment with tamoxifen (1 μM) for 16 hours (as established for CD4 downregulation—see e.g., Walk S. F. et al., Design and use of an inducibly activated human immunodeficiency virus type 1 Nef to study immune modulation. J VIROL 2001; 75 (2):834-843), fluorescence cytometry analysis was performed with an anti-S1P1 monoclonal antibody coupled to eFluor 660 (SW4GYPP, EBIOSCIENCE™). Results demonstrated that Nef activation downregulated S1P1 surface expression only in Nef-ER cells but not in control T cell line (SupT1) treated also with tamoxifen. To determine whether there was a specific pathway involved in HIV-Nef dependent S1P1, downregulation, PAK2 signaling was tested. As shown in FIG. 1A, PAK2 inhibition with 5 μM FRAX597 showed that the PAK2 inhibitor not only restored S1P1 surface expression, but also increased S1P1 surface expression when Nef was activated.

In contrast, PAK2 inhibition in mock transfected cells showed downregulation of S1P as reported previously (see, e.g., Pene-Dumitrescu T. et al. HIV-1 Nef interaction influences the ATP-binding site of the Src-family kinase, Hck., BMC CHEM BIOL 2012, 12:1). This downregulation can lead to reduced chemotactic migration towards S1P as evidenced from experiments when 4 HT activated. SupT1I control and Nef-ER cells were tested for migration through collagen I-coated transwell chambers towards S1P gradients as previously described for Jurkat T cells (Iino J. et al. 2015). As shown in FIG. 1B, a reduced number of Nef-ER cells migrated to the bottom chamber containing 100 nM S1P over 4 hours at 37° C.; however, pretreatment of PAK2 inhibitor not only restored the effect of NEF-ER but also showed significant increase in migration. Taken together, these results show that HIV-Nef activation causes S1P1 downregulation and reduced migration to S1P, which can be restored to values that is higher than that observed in control T cells.

Referring now to FIG. 1D, Nef activation upon treatment with tamoxifen downregulated S1P1 surface expression only in Nef-ER cells but not in control T cells (SupT1). PAK2 inhibition (5 μM FRAX 597, TOCRIS) restored S1P1 surface expression with Nef activation. In contrast, PAK2 inhibition in mock transfected cells showed downregulation of S1P1 as reported previously (see e.g., Phee H. et al., Pak2 is required for actin cytoskeleton remodeling, TCR signaling, and normal thymocyte development and maturation. ELIFE 2014; 3:e02270). These results confirm that HIV-Nef mediated. S1P1 downregulation and degradation can be a target mechanism to explain retention of HIV-infected and Nef expressing latent CD4 positive T cells and monocytic cells.

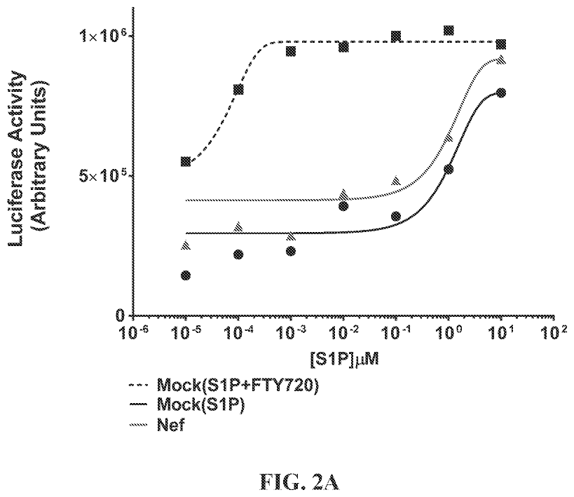

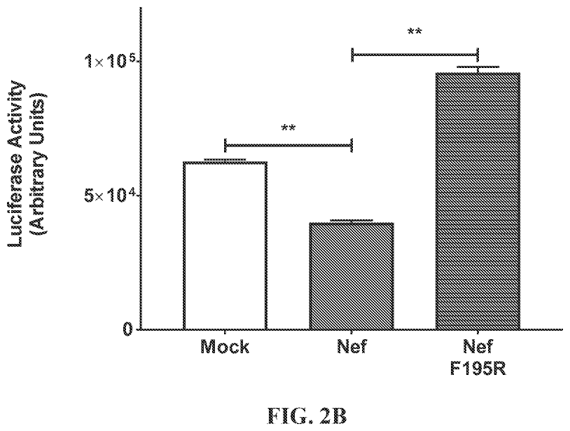

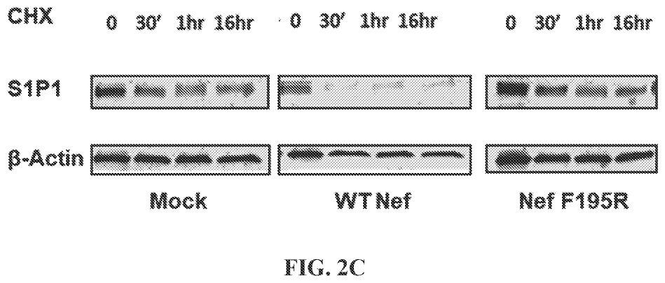

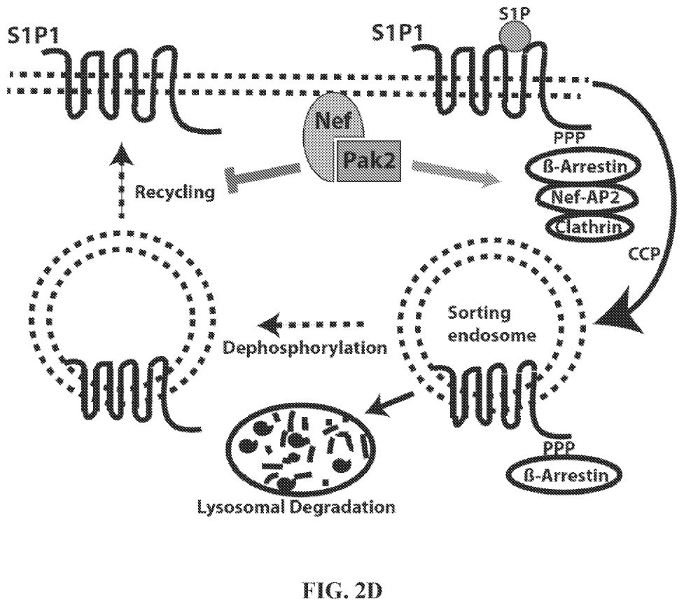

HIV-Nef Increases β-Arrestin Recruitment and Destabilization of S1P1. The effect of Nef expression on β-arrestin recruitment was examined. β-arrestin recruitment is a central mechanism for G protein coupled receptor (GPCR) recycling and degradation. Briefly, the S1P1-Tango expression vector (Addgene) was expressed in HTLA cells (HEK293) stably expressing β-arrestin-TEV protease fusion protein and the Tet-luciferase gene (see e.g., Inagaki H. K. et al., Visualizing neuromodulation in vivo: TANGO-mapping of dopamine signaling reveals appetite control of sugar sensing. CELL 2012; 148 (3):583-595). Upon activation of S1P1 by specific ligands (e.g. S1P or fingolimod), β-arrestin is recruited to the C-terminus of the receptor. This is followed by cleavage of the C-terminally fused Tet-activator at a TEV protease recognition site. The released tTA transcriptional activator, after transport to the nucleus, activates transcription of the luciferase reporter gene. HTLA cells were first transfected with S1P1-Tango and 20 hours later with either HIV-SF2-Nef (“Nef”) or the PAK2 activation deficient mutant F195R (“Nef F195R”). Luciferase activity was measured in response to S1P. Using the Tango GPCR activation model, increased β-arrestin recruitment were observed in Nef co-transfected HTLA cells in a dose response to S1P when compared to mock transfected cells (FIG. 2A).

Increased β-arrestin recruitment was observed in Nef and Nef F195R co-transfected HTLA cells. Nef F195R led to higher level of β-arrestin recruitment than that of Nef alone. It was unexpected for the Nef signaling mutant Nef F195R to show more β-arrestin recruitment than wild type Nef. However, these data can be explained by assessing the function of Nef in S1P1 internalization and endosomal recycling. Nef promotes cytoskeletal engagement in endocytosis. Inhibition of Nef-induced cytoskeletal engagement in endocytosis, through the inhibition of the Nef-PAK2 interface, will lead to the stabilization of S1P1 by inducing impaired trafficking to endosomal/lysosomal compartments. Further, the dose response to S1P in the presence of a constant concentration (1 μM) of the S1P receptor agonist FTY720 leads to a strong increase in β-arrestin recruitment, which suggests similar mechanisms involving S1P1 recycling and degradation.

Referring now to FIG. 2B, HTLA cells were concomitantly transfected with either S1P1-Tango and Nef, or S1P1-Tango and Nef F195R. Luciferase activity was measured in response to S1P after 2 days. Nef decreased recruitment of β-arrestin in response to S1P (black bar) and the decreased recruitment of β-arrestin was abolished when the PAK2 activation mutant was employed (e.g., Nef F195R), While not wishing to be bound by any theory, this unexpected finding can be due to prolonged interaction between Nef-PAK2 interface and S1P1, which can lead to S1P1 degradation through enhanced trafficking to endosomal/lysosomal compartments.

To further address this hypothesis, the same co-transfected HTLA cells were further examined in the presence of the protein synthesis inhibitor cycloheximide (50 μM). As shown in FIG. 1D, a continuous supply of overexpressed TANGO S1P1 in HTLA cells was abolished by the treatment of the protein synthesis inhibitor cycloheximide (50 μM). S1P1 degradation was observed in Nef expressing cells, but not in Nef F195R expressing cells in presence of the protein synthesis inhibitor cycloheximide (50 μM) after S1P stimulation.

The results disclosed herein demonstrate a basis for testing small molecular weight inhibitors targeting S1P1 downregulation in response to HIV-Nef (FIG. 2D). Based on the complexity of the findings, initial drug screening can involve all three test systems tested in this disclosure (e.g., β-arrestin recruitment, protein stability, and surface expression). The results further provide that HIV-Nef functions similar to fingolimod through desensitization and degradation of the S1P1 receptor. Of note, the dose response to S1P in the presence of a constant concentration of 1 μM FTY720 led to a strong synergistic increase in β-arrestin recruitment, suggesting involvement of distinct and possibly complementary mechanisms that govern HIV-Nef- and fingolimod-induced S1P1 recycling and degradation.

Based on the findings presented here, initial target identification could involve migration towards S1P to assess functional activity of S1P1. Further, the presently established assay for testing HIV-Nef signaling in S1P1 downregulation include an in vitro migration assay for T cells though collagen coated filters, a TANGO® assay developed to study GPCR receptor regulation. These could be a basis for testing small molecular weight inhibitors targeting S1P1 downregulation in response to HIV-Nef. Based on the use of a specific Nef-signaling mutant, targeting the Nef-PAK2 interface can become a first promising druggable target. It is noted that the use of existing chemical PAK2 inhibitors can present one or more side effects based on the wide range of biological activities exerted by PAK2 that are essential for cytoskeletal movement, nuclear signaling, TCR signaling and T cell maturation (see e.g., Phee H. et al., Pak2 is required for actin cytoskeleton remodeling, TCR signaling, and normal thymocyte development and maturation. ELIFE 2014; 3:e02270). Accordingly, Nef-PAK2 interface can be a therapeutic target for treating a subject having HIV-infection or a related clinical condition.

The data presented herein provides that HIV-Nef, the HIV encoded protein shown to persist in patients on successful antiretroviral therapy, can downregulate S1P1, which is a central regulator of T cell egress from lymph nodes, and can be successfully targeted to treat autoimmune diseases. Consistently, impaired S1P mediated signaling in HIV-1 infected lymph nodes has been reported. Further, several in vivo models such as humanized mice (BLT) for HIV research can utilize the instant disclosure for treating HIV in preclinical models. In fact, using this BLT mouse model led to the successful identification Nef as a mechanism to enhance HIV-1 replication and deplete CD4+CD8+thymocytes (see, e.g., Zou W. et al. Nef functions in BLT mice to enhance HIV-1 replication and deplete CD4+CD8+ thymocytes. RETROVIROLOGY 2012, 9:44). Taken together, successful interference with HIV-Nef induced S1P1 downregulation can emerge as a novel therapy to enhance effects of antiretroviral therapy, “shock and kill” approaches, and immunization against HIV.

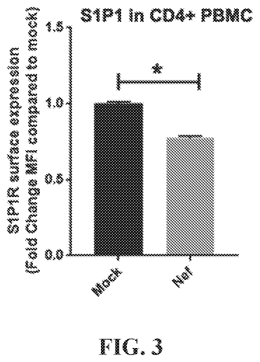

HIV-Nef promotes Surface Downregulation and Degradation of S1P1 Receptor: Implication for HIV Reservoirs. HEK293 cells were transfected with mock (mCherry containing plasmid) or Nef containing plasmid. EVs were isolated by ultracentrifugation at 100,000 g for 1 hours from supernatants harvested 24-48 hours after transfection and added to peripheral blood mononuclear cells (PBMC) isolated via ficoll gradient centrifugation. Surface S1P1 expression was determined by flow cytometry after staining with specific antibodies. To address for the known ability of Nef to downregulate CD4, PBMC were gated for CD4 dim cells prior to assessment of S1P1 positivity. Referring now to FIG. 3, HIV-Nef leads to S1P1 downregulation in CD⁺ PBMC.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety to the extent they are not inconsistent with the explicit teachings of this specification.

1. A composition, comprising:
at least one agent that inhibits Nef-PAK2 interface;
at least one agent that inhibits Nef-AP2 interface; and/or
at least one agent that inhibits PAK2-AP2-□-arrestin interface in a subject.

2. The composition according to claim 1, wherein the agent that inhibits Nef-PAK2 interface disrupts conformation of the Nef-PAK2 interface, and/or wherein the agent that inhibits Nef-AP2 interface disrupts conformation of the Nef-AP2 interface.

3. The composition according to claim 1, wherein the agent that inhibits Nef-PAK2 interface and/or Nef-AP2 interface is selected from at least one Nef inhibitor, at least one PAK2 inhibitor, at least one AP2 inhibitor, protein phosphatase 2 alpha, and combinations thereof.

4. The composition according to claim 1, further comprising at least one antiretroviral agent.

5. The composition according claim 4, wherein the antiretroviral agent is selected from zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir, lopinavir, indinavir, nelfinavir, amprenavir, ritonavir, darunavir, atazanavir, bevirimat, vivecon, and combinations thereof.

6. The composition according to claim 1, wherein the agent that inhibits Nef-PAK2 interface comprises a PAK2 inhibitor selected from FRAX1036, FRAX597, miR-23b, miR-137, and combinations thereof.

7. The composition according to claim 1, further comprising at least one agent that inhibits □-arrestin recruitment to at least one sphingosine-1-phosphate receptor, wherein the sphingosine-1-phosphate receptor is selected from Sphingosine-1-Phosphate Receptor 1 (S1P1), Sphingosine-1-Phosphate Receptor 2 (S1P2), Sphingosine-1-Phosphate Receptor 3 (S1P3), Sphingosine-1-Phosphate Receptor 4 (S1P4), Sphingosine-1-Phosphate Receptor 5 (S1P5), and combinations thereof.

8. The composition according to claim 1, wherein the subject is a human, an animal, a tissue, or a cell.

9. The composition according to claim 1, wherein the subject has been infected by human immunodeficiency virus (HIV).

10. The composition according to claim 1, wherein the subject has been treated with an antiretroviral agent selected from zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir lopinavir, indinavir, nelfinavir, amprenavir, ritonavir, darunavir, atazanavir, bevirimat, vivecon, and combinations thereof.

11. A method of treating an HIV infection, comprising:
administering to a subject a therapeutically effective dose of the composition according to claim 1 or a pharmaceutically acceptable salt or metabolite thereof.

12. The method according to claim 11, wherein the subject is a human, an animal, a cell, or a tissue.

13. The method according to claim 1, wherein the HIV infection comprises conditions and/or diseases associated with an HIV-infection, acquired immunodeficiency syndrome (AIDS), or a combination thereof.

14. The method according to claim 11, wherein the composition is administered orally or intravenously.

15. A method of reducing a side effect of a therapeutic regime, comprising:
administering to a subject at least one therapeutically effective dose of an agent that inhibits Nef-PAK2 interface and/or Nef-AP2 interface in a subject, wherein:
the subject has received at least one therapeutic regime selected from surgery, antiretroviral therapy (ART), highly active antiretroviral therapy (HAART) or a combination thereof, and
the subject is experiencing at least one side effect as a consequence of the therapeutic regime.

16. The method according to claim 15, wherein the agent that inhibits Nef-PAK2 interface and/or Nef-AP2 interface comprises at least one Nef inhibitor, at least one PAK2 inhibitor, at least one AP2 inhibitor, protein phosphatase 2 alpha, at least one agent that inhibits □-arrestin recruitment to at least one sphingosine-1-phosphate receptor, or a combination thereof.

17. The method according to claim 15, wherein the agent that inhibits Nef-PAK2 interface comprises at least one agent that inhibits □-arrestin recruitment to at least one sphingosine-1-phosphate receptor, wherein the sphingosine-1-phosphate receptor comprises Sphingosine-1-Phosphate Receptor 1 (S1P1), Sphingosine-1-Phosphate Receptor 2 (S1P2), Sphingosine-1-Phosphate Receptor 3 (S1P3), Sphingosine-1-Phosphate Receptor 4 (S1P4), and/or Sphingosine-1-Phosphate Receptor 5 (S1P5).

18. The method according to claim 15, wherein the subject has been treated with at least one antiretroviral agent selected from zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir lopinavir, indinavir, nelfinavir, amprenavir, ritonavir, darunavir, atazanavir, bevirimat, vivecon, and combinations thereof.

19. The method according to claim 15, wherein the side effect is selected from drug-resistance, relapse, retention of HIV-infected lymphocytes, generation of a viral reservoir, and combinations thereof.

20. The method according to claim 15, wherein the subject is a human or a non-human animal.
